(19) United States
(12) Patent Application Publication
(10) Pub. No.: US 2010/0158838 A1
(43) Pub. Date: Jun. 24, 2010

(54) NAIL VARNISH COMPRISING A SICCATIVE OIL AND A METAL SALT

(75) Inventor: Guillaume KERGOSIEN, Chaville (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 12/634,226

(22) Filed: Dec. 9, 2009

Related U.S. Application Data
(60) Provisional application No. 61/153,321, filed on Feb. 18, 2009.

(30) Foreign Application Priority Data
Dec. 17, 2008 (FR) 08 58694

(51) Int. Cl. A61K 8/97; A61Q 3/02

(52) U.S. Cl. 424/61

(57) ABSTRACT

The present invention relates to a nail varnish, characterized in that it comprises at least one siccative oil and at least one metal salt, and in that it comprises less than 5% by weight of volatile organic solvent(s) relative to the total weight of the nail varnish.

The present invention also relates to a non-therapeutic cosmetic process for making up and/or caring for the nails, comprising the application to the nails of at least one coat of the said nail varnish.

The present invention relates to a nail varnish comprising at least one siccative oil and at least one metal salt, and comprising less than 5% by weight of volatile organic solvent(s) relative to the total weight of the nail varnish.

This nail varnish composition may be applied to human nails or to false nails.

The present invention also relates to the corresponding non-therapeutic process for making up and/or caring for the nails.

The coloured or transparent nail varnish composition may be employed as a varnish base or “base coat”, as a nail makeup product, as a finishing composition, also known as a “top coat”, to be applied over the nail makeup product, or as a cosmetic nailcare product.

Nail varnish compositions traditionally comprise compounds dissolved in volatile organic solvents. These compositions form films after evaporation of the solvent.

However, these compositions have the drawback of releasing, during drying, odorous volatile compounds, which may be a nuisance to the user or people in the vicinity.

There is thus still a need for nail varnish compositions comprising a low content of volatile organic solvent(s), or even being free of volatile organic solvent.

The inventors have discovered that it is possible to obtain such a nail varnish composition comprising less than 5% by weight of volatile organic solvent(s) relative to the total weight of the composition, or even being free of the same, by using at least one siccative oil, preferably at least one refined or blown linseed oil, and at least one metal salt, which is preferably free of cobalt.

Thus, according to one of its aspects, one subject of the present invention is a nail varnish comprising at least one siccative oil, preferably at least one refined or blown linseed oil, at least one metal salt, which is preferably free of cobalt, and comprising less than 5% by weight of volatile organic solvent(s) relative to the total weight of the nail varnish, and preferably being free of volatile organic solvent.

Such a nail varnish especially has good stability over time, and allows the formation of a uniform, glossy and/or non-brittle film.

According to another aspect, a subject of the invention is also a non-therapeutic cosmetic process for making up and/or caring for the nails, comprising the application to the nails of at least one coat of a nail varnish as defined previously.

According to yet another of its aspects, a subject of the invention is also the use of a nail varnish as defined previously for obtaining, after deposition on the nail, a uniform, glossy and/or non-brittle film.

The nail varnish according to the invention comprises a cosmetically acceptable medium, i.e. a non-toxic medium that can be applied to human keratin materials, in particular the nails.

The present invention also relates to a made-up synthetic support comprising a makeup obtained via the process of the invention.

The nail varnish according to the invention has a liquid or gelled texture: it differs in particular from a patch or flexible article comprising at least one coat of polymer material and an adhesive coat, intended to be bonded and adjusted, by means of its deformability properties, onto the nail.

In other words, the nail varnish according to the invention is typically applied in the form of coats superposed onto the surface of the nails or false nails to be made up, for example using a brush.

A nail varnish according to the invention in particular comprises less than 5% by weight or even less than 2% by weight of volatile organic solvent(s), or even is free of volatile organic solvent.

The term “volatile organic solvent” means an organic solvent (or non-aqueous medium) that is capable of evaporating on contact with the nail in less than one hour, at room temperature and atmospheric pressure. Such an organic solvent is a volatile cosmetic solvent, which is liquid at room temperature, especially having a non-zero vapour pressure, at room temperature and atmospheric pressure, in particular having a vapour pressure ranging from 0.13 Pa to 40 000 Pa (10⁻³ to 300 mmHg) and preferably ranging from 1.3 Pa to 8000 Pa (0.01 to 60 mmHg).

These organic solvents are especially those conventionally used in nail varnishes. Among these solvents, examples that may be mentioned include ketones, for instance acetone, methyl ethyl ketone and methyl isobutyl ketone; glycol ethers; alcohols, for instance ethanol, n-butanol, n-propanol and isopropanol; acetates, for instance butyl, ethyl, isopropyl or 2-methoxyethyl acetate; linear or branched hydrocarbons such as hexane or octane; aromatic hydrocarbons such as xylene and toluene, or alternatively volatile silicone oils.

Siccative Oils

A nail varnish according to the invention comprises at least one siccative oil.

The term “siccative oil” is intended to denote an oil which, when spread as a thin coat and then exposed to the air, transforms into a solid film.

In particular, in the context of the present invention, the term “siccative oil” is intended to denote oils, and preferably triglycerides, comprising conjugate double bonds, preferably comprising at least two conjugate double bonds and preferably comprising at least three conjugate double bonds.

The siccative oils in accordance with the invention may be of natural origin.

Advantageously, the siccative oil may be chosen from siccative plant oils such as linseed oil, Chinese (or Cantonese) wood oil, oiticica oil, vernonia oil, poppy oil, pomegranate oil or marigold oil; esters of these plant oils; alkyd resins obtained from these plant oils; and mixtures thereof.

Alkyd resins are polyesters comprising fatty acid hydrocarbon chains, obtained especially by polymerization of polyols and polyacids or the corresponding anhydride thereof, in the presence of fatty acids. These fatty acids are present, especially in the form of triglycerides, in the majority of natural oils, in particular such as the oils mentioned previously.

The siccative oil that is suitable for use in the present invention may be modified by chemical reaction.

In particular, it may be refined and/or partially polymerized. In this respect, mention may be made of blown oils and stand oils, and maleinized, epoxidized or boiled oils.

According to one particular embodiment, the siccative oil of the invention is different from epoxidized oils.

According to one particular embodiment of the invention, the siccative oil is a refined linseed oil.

An oil may be refined in particular in three successive steps.

The refined linseed oil according to the invention may thus result from a degumming step, in particular to obtain a mucilage-free oil, followed by a decolorizing step, in particular to bleach it, and then a neutralizing step.

According to one particular embodiment of the invention, the siccative oil of the invention is a linseed oil that has been modified according to at least one of the three steps mentioned previously, in other words an oil that has undergone either a degumming step, or a decolorizing step, or a neutralizing step, or a succession of these steps.

According to yet another particular mode of the invention, the siccative oil of the invention is a modified linseed oil that has undergone two steps among those considered previously.

According to one particular embodiment of the invention, the siccative oil is a blown linseed oil.

The blowing of an oil is characterized especially by polymerization of the said oil with atmospheric oxygen. The blown oil may be obtained in particular by blowing air through the heated oil.

According to one particular embodiment, the siccative oil of the invention is different from an epoxidized oil.

According to one particular embodiment, the cosmetic nail varnish composition is according to the invention comprise from 40% to 99.9% by weight, especially from 60% to 99% by weight and in particular from 65% to 97% by weight of siccative oil relative to the total weight of the composition.

Metal Salts

A nail varnish in accordance with the invention comprises at least one metal salt and preferably at least two different metal salts.

The combination of at least one siccative oil with one (or more) metal salt(s) makes it possible especially on drying to form a film via crosslinking with the atmospheric oxygen.

The metal salt(s) in particular accelerate(s) the drying of the natural siccative oils.

Advantageously, the metal salt used comprises less than 5% of cobalt relative to the total weight of the metal in the metal salts, or even is advantageously free of cobalt, in particular so as to obtain a more favourable toxicological profile.

According to one particular embodiment, the metal salt(s) in accordance with the invention is (are) chosen from manganese, calcium, zirconium, zinc, strontium, lithium, cerium and vanadium salts.

According to one particular embodiment, the metal salt is an organic salt. It may especially be of formula (I) below:

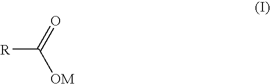

in which:

M is chosen from manganese, calcium, zirconium, zinc, strontium, lithium, cerium and vanadium; and

R is a linear or branched (C₁-C₂₅)alkyl radical, optionally comprising 1 to 3 unsaturations.

According to one particular mode, R is a linear or branched (C₆-C₂₀)alkyl radical, optionally comprising 1 to 2 unsaturations.

According to one particular mode, the metal salt may take the form of octoate, linoleate, octanoate, oleate, stearate, laurate or naphthenate.

According to one particular embodiment, a nail varnish in accordance with the invention comprises at least one manganese salt.

According to another particular embodiment of the invention, the nail varnish composition according to the invention comprises several metal salts, in particular for the purpose of increasing the rate of crosslinking of the siccative oil.

According to one particular embodiment of the invention, the cosmetic nail varnish compositions according to the invention comprise from 0.001% to 2% by weight, especially from 0.01% to 1% by weight and in particular from 0.1% to 0.5% by weight of metals relative to the solids content.

Examples of commercial products of metal salts are given below: Octa-Silogen Manganese 6 HS, Octa-Silogen Zirconium 12 HS and Octa-Silogen Calcium 5 HS, sold by the company OMG Borchers GMBH.

The indication xHS (6 HS, 12 HS and 5 HS) employed above indicates that the commercial product or starting material contains x percent of metal relative to the total weight of the commercial product or metal salt.

According to one particular mode of the invention, a nail varnish according to the invention may comprise at least two different metal salts chosen from the salts as defined previously.

According to another particular mode of the invention, the metal salts of a nail varnish of the invention may comprise at least one primary metal salt and at least one secondary metal salt, or even may consist of a primary metal salt and a secondary metal salt.

In the context of this particular embodiment:
the primary metal salt may be chosen from manganese salts, cerium salts and vanadium salts, and is more particularly a manganese salt; and
the secondary metal salt may be chosen from zirconium, zinc, lithium and strontium salts, and is more particularly a zirconium salt.

In the context of this particular embodiment, the metal salts of a nail varnish of the invention may comprise at least one manganese salt and at least one zirconium salt.

According to yet another preferred embodiment of the invention, a nail varnish of the invention may comprise at least one primary metal salt as defined previously, at least one secondary metal salt as defined previously, and at least one auxiliary metal salt.

In the context of the latter particular embodiment, the auxiliary metal salt may be chosen from calcium salts.

According to one particularly preferred embodiment, the metal salts of a nail varnish of the invention may comprise at least one manganese salt, at least one zirconium salt and at least one calcium salt, or even may consist of a manganese salt, a zirconium salt and a calcium salt.

In the context of this particularly preferred embodiment, the manganese salt may especially promote the surface drying and the complete drying of the film; the zirconium salt may promote the deep drying, inducing uniform drying of the film; and the calcium salt may make it possible to develop a synergistic effect between the manganese salt and the zirconium salt.

Still according to this particularly preferred embodiment, the nail varnish composition of the invention may comprise at least one primary metal salt as defined previously in a content ranging from 0.005% to 0.1% by weight of metal relative to the solids content and especially from 0.01% to 0.05% by weight of metal relative to the solids content. It may also comprise at least one secondary metal salt as defined previously in a content ranging from 0.005% to 0.8% by weight of metal relative to the solids content and especially from 0.03% to 0.5% by weight of metal relative to the solids content. Finally, it may comprise at least one auxiliary metal salt as defined previously, in a content ranging from 0.03% to 0.3% by weight of metal relative to the solids content.

Characterization of the Solids Content

The dry matter content, commonly known as the “solids content”, i.e. the content of non-volatile matter, may be measured in various ways. Mention may be made, for example, of oven-drying methods, and drying methods via exposure to infrared radiation.

Preferably, the solids content of the nail varnish compositions according to the invention is measured by heating the sample with infrared rays of wavelength 2 μm to 3.5 μm. The substances contained in the said compositions that have a high vapour pressure evaporate under the effect of this radiation. Measurement of the weight loss of the sample makes it possible to determine the “solids content” of the composition. These measurements are performed using an LP 16 commercial infrared desiccator from Mettler. This technique is fully described in the machine documentation supplied by Mettler.

The measuring protocol is as follows:

About 1 g of the composition is spread onto a metal crucible. After placing it in the desiccator, this crucible is subjected to a nominal temperature of 120° C. for one hour. The wet mass of the sample, corresponding to the initial mass, and the dry mass of the sample, corresponding to the mass after exposure to the radiation, are measured using a precision balance.

The solids content is calculated in the following manner:

solids content=100×(dry mass/wet mass)

The compositions according to the invention are characterized by a solids content of greater than or equal to 95% by weight and more particularly greater than 99% by weight.

In the particular case in which the composition is totally free of volatile organic solvent, the solids content may be equated to the weight of the total nail varnish composition.

Film-Forming Agent

A nail varnish according to the invention may also comprise a film-forming agent. The film-forming agent is generally a film-forming polymer.

For the purposes of the present invention, the term “film-forming polymer” denotes a polymer that is capable, by itself or in the presence of an auxiliary film-forming agent, of forming an isolable and especially continuous and adherent film, on a support, especially on the nails.

In particular, the film-forming polymer of the invention is different from the siccative oil as above-described.

In the composition, a single film-foaming polymer or a mixture of film-forming polymers may be used.

This film-forming polymer may be chosen from the group formed by synthetic polymers, of free-radical type or of polycondensate type, polymers of natural origin, and mixtures thereof.

A film-forming polymer that is suitable for use in the invention may be chosen in particular from:
polysaccharide derivatives, such as cellulose or guar gum derivatives. A polysaccharide derivative that is suitable for use in the invention may be a polysaccharide ester or alkyl ether.

The term “polysaccharide ester or alkyl ether” denotes a polysaccharide formed from repeating units comprising at least two identical or different rings and having a degree of substitution per saccharide unit of between 1.9 and 3, preferably between 2.2 and 2.9 and more particularly between 2.4 and 2.8. The term “substitution” denotes the functionalization of the hydroxyl groups as ester and/or alkyl ether functions, and/or the functionalization of the carboxylic groups as ester functions.

In other words, it may be a polysaccharide that is partially or totally substituted with ester and/or alkyl ether groups. Preferably, the hydroxyl groups may be substituted with C₂-C₄ ester and/or alkyl ether functions.

Mention may be made in particular of cellulose esters, such as cellulose acetate, cellulose acetobutyrates or cellulose acetopropionates; cellulose alkyl ethers, for instance ethyl celluloses, and ethyl guars;
synthetic polymers such as polyurethanes, acrylic polymers, vinyl polymers, polyvinyl butyrals, alkyd resins and ketone/aldehyde resins, resins derived from aldehyde condensation products, such as arylsulfonamide-formaldehyde resins, for instance toluenesulfonamide-formaldehyde resin, arylsulfonamide-epoxy resins or ethyl tosylamide resins;
polymers of natural origin, such as plant resins, such as dammar resins, elemi gums, copal resins, and benjoin; gums such as shellac, sandarac gum and mastic gum.

Film-forming polymers that may especially be used include the toluenesulfonamide-formaldehyde resins Ketjenflex MS80 from the company Akzo or Santolite MHP and Santolite MS 80 from the company Faconnier or Resimpol 80 from the company Pan Americana, the alkyd resin Beckosol ODE 230-70-E from the company Dainippon, the acrylic resin Acryloid B66 from the company Rohm & Haas, the polyurethane resin Trixene PR 4127 from the company Baxenden, and the acetophenone/formaldehyde resin sold under the reference Synthetic Resin SK from Degussa.

According to one preferred embodiment, the film-forming agent is chosen from plant resins and gums such as shellac, sandarac gum, dammar resins, elemi gum, copal resins, benjoin and mastic gum,

For example, the distilled, purified and crushed elemi gum sold by the company Emiga may be used as film-forming polymer.

According to one particular embodiment, a nail varnish according to the invention comprises less than 30% by weight, especially less than 20% by weight and in particular less than 10% by weight of film-forming agent relative to the total weight of the composition.

According to one preferred embodiment, a nail varnish composition according to the invention comprises less than 5% by weight, preferably less than 3% by weight, preferably less than 1.5% by weight and preferably less than 1% by weight of nitrocellulose solids relative to the total weight of the composition.

According to one particularly preferred embodiment, the nail varnish composition of the invention is totally free of nitrocellulose.

According to one particular embodiment of the invention, a nail varnish according to the invention comprises at least one blown linseed oil and at least manganese, zirconium and calcium salts.

According to yet another particular mode of the invention, a nail varnish according to the invention also comprises elemi gum.

Auxiliary Film-Forming Agent

To improve the film-forming properties of the nail-varnish composition, an auxiliary film-forming agent may be envisaged.

Such an auxiliary film-forming agent may be chosen from any compound known to those skilled in the art as being capable of fulfilling the desired function, and may be chosen especially from plasticizers and coalescers for the film-forming polymer(s).

Thus, the composition may also comprise at least one plasticizer and/or coalescer. In particular, mention may be made, alone or as a mixture, of the usual plasticizers and coalescers, such as:
esters of acids, especially of carboxylic acids, such as citrates, phthalates, adipates, carbonates, tartrates, phosphates and sebacates;
oxyethylenated derivatives, such as oxyethylenated oils, especially plant oils, such as castor oil;
oils of natural origin, in particular non-siccative oils, chosen from oils comprising at least one fatty acid chosen from caprylic acid, capric acid, lauric acid, myristic acid, palmitic acid, stearic acid, oleic acid, ricinoleic acid, linoleic acid, linolenic acid, arachidic acid, gadoleic acid, behenic acid, erucic acid, brassidic acid, cetoleic acid, lignoceric acid and nervonie acid. In particular, these oils are chosen from triglycerides formed from fatty acid esters of glycerol in which the fatty acids may have chain lengths ranging from C₄ to C₂₄, these chains possibly being linear or branched, and saturated or unsaturated. These oils are especially heptanoic or octanoic triglycerides, groundnut oil, babassu oil, coconut oil, grape seed oil, cotton seed oil, corn oil, corn germ oil, mustard seed oil, palm oil, sesame seed oil, soybean oil, sunflower oil, wheatgerm oil, canola oil, apricot oil, mango oil, shea oil, avocado oil, olive oil, sweet almond oil, almond oil, peach oil, walnut oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil, musk rose oil, shea butter oil or caprylic/capric acid triglycerides, and
mixtures thereof.

The type and amount of plasticizer(s) and/or coalescer may be chosen by a person skilled in the art on the basis of his general knowledge.

For example, the content of plasticizer and/or coalescer may range from 0.01% to 20% and in particular from 0.5% to 10% by weight relative to the total weight of the composition.

Gelling Agent

The nail varnish composition according to the invention may also comprise a gelling agent.

This gelling agent may be chosen in particular from: hydrophobic silicas, such as those described in document EP-A-0 898 960, and sold, for example, under the references Aerosil R812® by the company Degussa, Cab-O-Sil TS-530®, Cab-O-Sil TS-610® and Cab-O-Sil TS-720® by the company Cabot, and Aerosil R972® and Aerosil R974® by the company Degussa; clays such as montmorillonite, modified clays such as bentones, for example stearalkonium hectorite and stearalkonium bentonite, and polysaccharide alkyl ethers (especially in which the alkyl group contains from 1 to 24, preferably from 1 to 10, better still from 1 to 6 and more especially from 1 to 3 carbon atoms) such as those described in document EP-A-0 898 958.

The total proportion of gelling agent(s) in the compositions according to the invention may range from 0.01% to 15% by weight, preferably from 0.5% to 15% and better still from 0.5% to 10% by weight relative to the total weight of the composition.

Dyestuff

The nail varnish composition according to the invention may also comprise one or more dyestuffs chosen from soluble dyes and pulverulent dyestuffs such as pigments, nacres and glitter flakes that are well known to those skilled in the art.

The dyestuffs may be present in the composition in a content ranging from 0.01% to 50% by weight and preferably from 0.01% to 30% by weight relative to the weight of the composition.

The term “pigments” should be understood as meaning white or coloured, mineral or organic particles of any shape, which are insoluble in the physiological medium, and are intended to colour the composition.

The term “nacres” should be understood as meaning iridescent particles of any shape, especially produced by certain molluscs in their shell, or else synthesized.

The pigments may be white or coloured, and mineral and/or organic. Among the mineral pigments, mention may be made of titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders such as aluminium powder or copper powder.

Among the organic pigments, mention may be made of carbon black, pigments of D&C type, and lakes based on cochineal carmine or on barium, strontium, calcium or aluminium.

Mention may also be made of pigments with an effect, such as particles comprising a natural or synthetic, organic or mineral substrate, for example glass, acrylic, polyester, polyurethane, polyethylene terephthalate resins, ceramics or aluminas, and optionally coated with metal substances such as aluminium, gold, copper or bronze, or coated with metal oxides such as titanium dioxide, iron oxide, chromium oxide or mineral or organic pigments, and mixtures thereof.

The nacreous pigments may be chosen from white nacreous pigments such as mica coated with titanium or with bismuth oxychloride, coloured nacreous pigments such as titanium mica coated with iron oxides, titanium mica coated especially with ferric blue or with chromium oxide, titanium mica coated with an organic pigment of the abovementioned type, and also nacreous pigments based on bismuth oxychloride.

Pigments with goniochromatic properties, especially liquid crystals or multilayer crystals, may also be used,

The dyes are, for example, Sudan red, DC Red 17, DC Green 6, β-carotene, soybean oil, Sudan brown, DC Yellow 11, DC Violet 2, DC Orange 5 and quinoline yellow.

The dyestuff may also be chosen from optical brighteners.

The composition may also comprise fibres optionally coated with optical brighteners.

Filler

The composition according to the invention may also comprise one or more fillers, especially in a content ranging from 0.01% to 50% by weight and preferably ranging from 0.01% to 30% by weight relative to the total weight of the composition. The term “fillers” should be understood as meaning colourless or white, mineral or synthetic particles of any shape, which are insoluble in the medium of the composition irrespective of the temperature at which the composition is prepared. These fillers serve especially to modify the rheology or texture of the composition.

The fillers may be mineral or organic and of any shape, platelet-shaped, spherical or oblong, irrespective of the crystallographic form (for example lamellar, cubic, hexagonal, orthorhombic, etc.). Mention may be made of talc, mica, silica, kaolin, polyamides (Nylon®) powder (Orgasol® from Atochem), poly-β-alanine powder and polyethylene powder, powders of tetrafluoroethylene polymers (Teflon®), lauroyl lysine, starch, boron nitride, hollow polymer microspheres such as those of polyvinylidene chloride/acrylonitrile, for instance Expancel® (Nobel Industrie), or microspheres of acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

Additives

The composition may also comprise other ingredients commonly used in cosmetic compositions and known to those skilled in the art as being able to be incorporated into a nail varnish composition.

Such ingredients may be chosen from oils, waxes, free-radical scavengers, spreading agents, wetting agents, dispersants, antifoams, preserving agents, surfactants, fragrances, neutralizers, stabilizers, antioxidants, active agents, which may be chosen from essential oils, UV-screening agents/sunscreens, moisturizers, vitamins, proteins, ceramides, plant extracts, etc.; and mixtures thereof.

The compositions according to the invention may also comprise, as active agents, naileare agents such as keratin material hardeners, active agents that act on the growth of the nail, for instance methyl sulfonyl methane and/or active agents for treating various complaints localized on the nail, for instance onychomycosis.

Needless to say, a person skilled in the art will take care to select this or these optional additional compound(s), and/or the amount thereof, such that the advantageous properties of the composition for the use according to the invention are not, or are not substantially, adversely affected by the envisaged addition.

According to another aspect, a subject of the invention is a nail varnish product comprising: i) a container delimiting at least one compartment, the said container being closed by a closing member, and ii) a composition according to the invention placed inside the said compartment.

The container may be of any suitable shape. It may especially be in the form of a bottle and may be at least partly made of a material such as glass. However, materials other than glass may be used, for instance thermoplastics such as PP or PE, or a metal.

The closing member may be coupled to the compartment by screwing in the closed position of the container. Alternatively, the coupling between the closing member and the container may take place other than by screwing, especially by click-fastening.

The container is preferably equipped with an applicator, which may be in the form of a brush formed from at least one tuft of bristles. Alternatively, the applicator may be in a form other than a brush, for example in the form of a spatula or a foam tip.

The invention is illustrated in greater detail in the examples that follow, which are given as non-limiting illustrations of the invention.

Unless otherwise indicated, the amounts are given as weight percentages relative to the total weight of the composition.

### EXAMPLES

### Example 1

| | |
|---|---|
| Blown linseed oil “Lin Soufflé CV 5P”⁽¹⁾ | 96% |
| Manganese octoate⁽²⁾ | 0.6% |
| Zirconium octoate⁽³⁾ | 1.8% |
| Neutral calcium octoate⁽⁴⁾ | 1.6% |

⁽¹⁾sold by the company Novance
⁽²⁾Octa-Soligen Manganese 6 HS sold by the company OMG Borchers GMBH
⁽³⁾Octa-Soligen Zirconium 12 HS sold by the company OMG Borchers GMBH
⁽⁴⁾Octa-Soligen Calcium 5 HS sold by the company OMG Borchers GMBH

### Reference Example

### Without Metal Salts

| | |
|---|---|
| Blown linseed oil “Lin Soufflé CV 5P”⁽¹⁾ | 100% |

Procedure:

Films of these two compositions are coated to a wet thickness of 50 μm onto a contrast card.

Result:

After drying for 7 hours at 30° C., the composition of Example 1 forms a solid film, whereas the reference composition is still liquid.

### Example 2

| | |
|---|---|
| Blown linseed oil “Lin Soufflé CV 5P”⁽¹⁾ | 86.4% |
| Distilled, purified, crushed elemi gum⁽⁵⁾ | 9.6% |
| Manganese octoate⁽²⁾ | 0.6% |
| Zirconium octoate⁽³⁾ | 1.8% |
| Neutral calcium octoate⁽⁴⁾ | 1.6% |

⁽⁵⁾sold by the company Emiga

Procedure:

In a first stage, the blown linseed oil and the elemi gum are mixed together.

The mixture is stirred until dissolution of the elemi gum is complete. The metal salts are then added.

A film of the composition is coated to a wet thickness of 50 μm onto a contrast card.

Result:

After drying, the composition forms a solid film.

1. Nail varnish, comprising at least one siccative oil and at least one metal salt, and comprising less than 5% by weight of volatile organic solvent(s) relative to the total weight of the nail varnish.

2. Nail varnish according to claim 1, wherein the siccative oil is chosen from linseed oil, Chinese wood oil, oiticica oil, vernonia oil, poppy oil, pomegranate oil and marigold oil; esters of these plant oils; alkyl resins obtained from these plant oils, and mixtures thereof.

3. Nail varnish according to claim 1, wherein the siccative oil is refined or blown linseed oil.

4. Nail varnish according to claim 1, wherein the siccative oil is present in a content ranging from 40% to 99.9% by weight, relative to the total weight of the nail varnish.

5. Nail varnish according to claim 1, wherein the metal salt comprises at least one metal salt chosen from manganese, calcium, zirconium, zinc, strontium, lithium, cerium and vanadium salts.

6. Nail varnish according to claim 5, wherein the metal salt is an organic salt of formula (I) below: 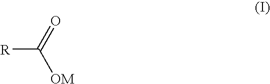
in which:
M is chosen from manganese, calcium, zirconium, zinc, strontium, lithium, cerium and vanadium; and
R is a linear or branched (C₁-C₂₅)alkyl radical, optionally comprising 1 to 3 unsaturations.

7. Nail varnish according to claim 1, said nail varnish comprising at least one manganese salt.

8. Nail varnish according to claim 1, wherein the metal salt comprises at least two different metal salts chosen from manganese, calcium, zirconium, zinc, strontium, lithium, cerium and vanadium salts.

9. Nail varnish according to claim 1, wherein the metal salt comprises at least one manganese salt and at least one zirconium salt.

10. Nail varnish according to claim 1, wherein the metal salt comprises at least one manganese salt, at least one zirconium salt and at least one calcium salt.

11. Nail varnish according to claim 1, wherein the metal salt(s) is (are) present in a content ranging from 0.001% to 2% by weight, relative to the solids content.

12. Nail varnish according to claim 1, said nail varnish comprising less than 2% by weight of volatile organic solvent(s) relative to the total weight of the nail varnish.

13. Nail varnish according to claim 1, said nail varnish being free of volatile organic solvent.

14. Nail varnish according to claim 1, said nail varnish also comprising at least one film-forming agent.

15. Nail varnish according to claim 14, wherein the film-forming agent is chosen from plant resins and gums.

16. Nail varnish according to claim 1, said nail varnish comprising less than 5% by weight of nitrocellulose solids.

17. Non-therapeutic cosmetic process for making up and/or caring for the nails, comprising the application to the nails of at least one coat of a nail varnish comprising at least one siccative oil and at least one metal salt, and comprising less than 5% by weight of volatile organic solvent(s) relative to the total weight of the nail varnish.
